(19) United States
(12) Patent Application Publication
(10) Pub. No.: US 2010/0130859 A1
(43) Pub. Date: May 27, 2010

(54) DETECTION OF VIABLE SPERM USING HIGH FREQUENCY ULTRASONIC IMAGING

(76) Inventor: Keith Jarvi, Toronto (CA)

Correspondence Address: CLARK & ELBING LLP, 101 FEDERAL STREET, BOSTON, MA, 02110, US

(21) Appl. No.: 12/447,948

(22) PCT Filed: Oct. 30, 2007
(86) PCT No.: PCT/CA07/01937

§ 371 Date: Feb. 2, 2010

Related U.S. Application Data
(60) Provisional application No. 60/855,269, filed on Oct. 30, 2006.

(51) Int. Cl. A61B 8/14

(52) U.S. Cl. 600/443

(57) ABSTRACT

Provided are methods and systems for detecting viable sperm in a subject having at least one testicle using high frequency ultrasound imaging. Also provided are high frequency ultrasound imaging systems and methods for screening a testicle for the presence of viable sperm, for predicting the success of vasectomy reversal in a subject, for identifying a damaged area of vas deferens in a subject, for detecting neoplasia in a subject having at least one testicle, for identifying a nerve in a subject, and for identifying small vessel anatomy in the penis of a subject.

### CROSS REFERENCE TO RELATED APPLICATIONS

This application claims the benefit of U.S. Provisional Application No. 60/855,269, filed Oct. 30, 2006, which is hereby incorporated by reference in its entirety.

### SUMMARY OF THE INVENTION

Provided are methods and systems for detecting viable sperm in a subject having at least one testicle. Also exemplarily provided are systems and methods for screening a testicle for the presence of viable sperm, for predicting the success of vasectomy reversal in a subject, for identifying a damaged area of vas deferens in a subject, for detecting neoplasia in a subject having at least one testicle, for identifying a nerve in a subject, and for identifying small vessel anatomy in the penis of a subject.

Other systems, methods, aspects and advantages of the invention will be discussed with reference to the Figures and to the detailed description of the preferred embodiments.

### BRIEF DESCRIPTION OF THE FIGURES

The accompanying drawings, which are incorporated in and constitute a part of this specification, illustrate several aspects described below and together with the description, serve to explain the principles of the invention.

FIG. 1 is a block flow diagram illustrating an exemplary method for detecting viable sperm in a subject having at least one testicle.

FIG. 2 is a block flow diagram illustrating an exemplary method for identifying a damaged area of vas deferens in a subject.

FIG. 3 is a block flow diagram illustrating an exemplary method for detecting neoplasia in a subject having at least one testicle.

FIG. 4 is a block flow diagram illustrating an exemplary method for identifying a nerve in a subject.

FIG. 5 is a block flow diagram illustrating an exemplary method for identifying small vessel anatomy in the penis of a subject.

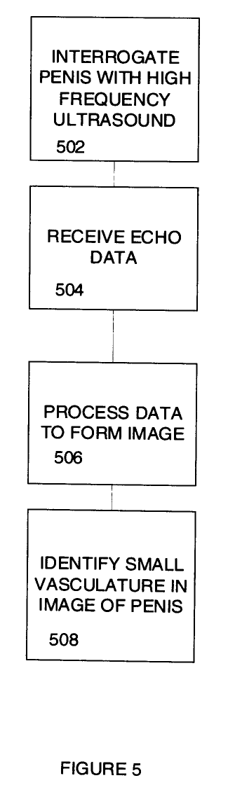

FIG. 6 is an exemplary ultrasonic image produced using the methods and systems described herein.

FIG. 7 is an exemplary ultrasonic image produced using the methods and systems described herein.

FIG. 8 is an exemplary ultrasonic image produced using the methods and systems described herein.

FIG. 9 is an exemplary ultrasonic image produced using the methods and systems described herein.

### DETAILED DESCRIPTION OF THE INVENTION

The present invention can be understood more readily by reference to the following detailed description, examples, drawing, and claims, and their previous and following description. However, before the present systems and/or methods are disclosed and described, it is to be understood that this invention is not limited to the devices systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” include plural referents unless the context clearly dictates otherwise. Thus, for example, reference to a “testicle” can include two or more such testicles unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

By a “subject” is meant an individual. The term subject includes small or laboratory animals as well as primates, including humans. A laboratory animal includes, but is not limited to, a rodent such as a mouse or a rat. The term laboratory animal is also used interchangeably with animal, small animal, small laboratory animal, or subject, which includes mice, rats, cats, dogs, fish, rabbits, guinea pigs, rodents, etc. The term laboratory animal does not denote a particular age or sex. Thus, adult and newborn animals, as well as fetuses (including embryos), whether male or female, are included. The term “patient” includes human and veterinary patients.

Approximately 7.5% of men are infertile and are candidates for intracytoplasmic sperm injection, a relatively new procedure which allows men who have no sperm in their ejaculate, but have a small number of sperm in their testes, to have their sperm extracted and injected directly into their partner's eggs during in vitro fertilization or intracytoplasmic sperm injection. This procedure often results in a pregnancy.

Under current clinical procedures, multiple and large biopsies are taken from the male patient resulting in severe scarring and devascularization. However, in one embodiment, the exemplary methods described herein utilize high frequency ultrasound to identify seminiferous tubules. In some examples, the large, for example, the largest, identified seminiferous tubule or tubules contain live sperm. In contrast to the current clinical procedures, sperm can then be obtained either using a very small biopsy or through ultrasound-guided needle aspiration.

Thus, provided are methods and systems for detecting viable sperm in a subject having at least one testicle. An exemplary method can comprise percutaneously interrogating at least a portion of a subject's testicle by transmitting sound having a frequency of at least about 15 megahertz (MHz) into the testicle. The exemplary method can further comprise receiving echo data from the interaction of the transmitted sound and the testicle, processing the received data to form an image, and detecting at least one viable sperm in the image. A viable sperm detected in the image can indicate viable sperm in the subject having at least one testicle.

Also exemplarily provided are systems and methods for screening a testicle for the presence of viable sperm, for predicting the success of vasectomy reversal in a subject, for identifying a damaged area of vas deferens in a subject, for detecting neoplasia in a subject having at least one testicle, for identifying a nerve in a subject, and for identifying small vessel anatomy in the penis of a subject.

In one aspect, the systems and methods can be used to detect the presence of and to quantify sperm production in a subject. Such systems and methods can be non-invasive and applied percutaneously. High frequency percutaneous ultrasound imaging can be used for direct visualization of motile and immotile sperm. Moreover, the methods can be accomplished by indirect indications such as by detecting differences of the size of seminiferous tubules within the testis. For example, large tubules up to 300 microns in diameter or more in size can indicate the presence of sperm. Similarly, movement of fluid within the testis hilum can indicate of the presence of sperm. Also, areas of different vascular perfusion as shown by Doppler ultrasound can indicate of the presence of sperm. For example, Doppler imaging can be performed with or without perfusion agents and higher perfusion can indicate areas of spermatogenesis and can indicate of the presence of sperm.

In a further aspect, also provided are systems and methods for predicting and/or for enhancing the success of vasectomy reversal. Between 5-10% of the 600,000+ men annually who undergo a vasectomy will later choose to have the vasectomy reversed. Vasectomy reversal is a micro-surgical procedure performed using an operating microscope that requires up to 4 hours of operating time. The surgery typically involves reconnecting the vas deferens (the tube carrying sperm) and allowing the sperm to pass through once again. Success rates of vasectomy reversal surgery vary and conventionally result in pregnancy approximately 50% of the time. Failure of vasectomy reversal is often caused by damage to the vas deferens in locations that are remote from the ligation site. Exemplary methods described herein utilize high-frequency ultrasound to image the vas deferens. For example, the vas deferens can be imaged along its entire path, or a portion thereof, including sites remote from the ligation site to visualize any damaged areas that could potentially result in the failure of the surgery. When damaged areas are identified, these areas can be repaired to help ensure surgical success.

In yet another aspect of the present invention, the systems and methods can also be used to detect the presence and quantification of sperm in the vas deferens and/or epididymis. Moreover, the methods and systems can be used to identify areas of obstruction/absence of the epididymis and vas deferens. Currently available non-invasive methods to determine obstruction and the presence of sperm in the vas deferens and the epididymis are unreliable. This diagnosis is now performed at the time of surgery using an operative microscope. The described methods and systems can be used to identify areas of obstruction by direct visualization of the obstruction or visualization of dilated tubules proximal to the obstruction, and of sperm within the proximal tubules. The systems and methods allow for prediction before surgery of who might benefit most from the surgery and also to guide the surgery precisely to the area of obstruction. Up to 40% of men who have had a vasectomy have obstruction in the epididymis. Currently, this diagnosis is only made at the time of surgery. Knowledge of this prior to surgery using the described methods and systems allows for more accurate prognosis. The systems and methods are also used to guide the surgical approach which improves the outcome of the surgery.

In one exemplary aspect, the systems and methods can also be used for detection of testis cancer. Testicular cancer is most common neoplasia in young men.

There are limited non-invasive techniques available to detect cancer of the testis. The systems and methods can be used to detect/diagnose early cancer of the testis by direct visualization of abnormal areas or by detecting indirect indications of cancer such as, for example and without limitation, areas of altered vascularity, areas of changes in the architecture of the seminiferous tubules, and the like.

Moreover, for example and without limitation, the systems and methods can be used as a precise guide for percutaneous and open surgical procedures, for biopsies and aspirations of sperm and tissue (for both sperm production/presence and cancer diagnosis/detection), and for the precise instillation of diagnostic or therapeutic agents into specific small structure (e.g., intra-tubular instillation of therapeutic agents). Using the methods and systems, therapeutic agents (e.g., pharmacotherapy, gene therapy, cell transplantation) can be instilled directly into the tubules. Agents to obstruct the vas deferens for a minimally invasive vasectomy can also be instilled using the precise guidance provided by the methods and systems.

In another aspect, the methods and systems are also used to determine small vessel anatomy in the penis or clitoris of a subject. Erectile dysfunction may often be due to small vessel dysfunction. Unfortunately, available non-invasive methods to determine small vessel anatomy in the penis are limited. Thus, diagnosing this small vessel disease and following the changes in small vessels with therapy (e.g., following new angiogenic therapy) using the systems and methods of the present invention allows for monitoring and tailoring of therapy.

In a further aspect, the methods and systems can also be used to identify the nerves for erections prior to, during and after therapy/surgery for prostate/rectal/pelvic diseases. The methods and systems allow for precise electrical stimulation of these nerves, which can confirm the nerve's function. Moreover, nerves can be damaged during therapy for pelvic diseases. As an example, close to 80% of men have erectile changes following radical prostatectomies. Precise identification of these nerves for erections using the methods and systems of the present invention such as, for example and without limitation, visualization of nerves with or without the requirement for electrical stimulation, allows for a better ability to preserve important nerves or to consider a nerve graft if preservation of the nerves would compromise care.

As used herein, high frequency ultrasound refers to ultrasound of a sufficiently high frequency to accurately resolve the desired urologic structures described herein. In some aspects, such systems can transmit ultrasound at a center transducer frequency of about 15 MHz or higher. In exemplary aspects, ultrasound can be supplied at a center frequency about 15 MHz, 20 MHz, 25 MHz, 30 MHz, 35 MHz, 40 MHz, 45 MHz, 50 MHz, 55 MHz, 60 MHz, 65 MHz, 70 MHz, or higher, and a frequencies therebetween. The systems and methods can include use of a bandwith of between about 10 MHz and about 80 MHz. A high frequency ultrasound probe including a mechanical sector scan transducer or an array transducer can be used to deliver and to receive the ultrasound.

In operation, ultrasound images are normally formed by the analysis and amalgamation of multiple pulse echo events. An image is formed, effectively, by scanning regions within a desired imaging area using individual pulse echo events, referred to as “A-Scans” or ultrasound “lines.” Each pulse echo event requires a minimum time for the acoustic energy to propagate into the subject and to return to the transducer. An image is completed by “covering” the desired image area with a sufficient number of scan lines, referred to as “painting in” the desired imaging area so that sufficient detail of the subject anatomy can be displayed. The number of and order in which the lines are acquired can be controlled by the ultrasound system, which also converts the raw data acquired into an image. Using a combination of hardware electronics and software instructions in a process called “scan conversion,” or image construction, the ultrasound image obtained is rendered so that a user viewing the display can view the subject being imaged.

Ultrasound imaging systems can transmit pulsed energy along a number of different directions, or ultrasonic beams, and thereby receive diagnostic information as a function of both lateral directions across the body and axial distance into the body. This information can be displayed as two dimensional, “B-scan” images. Such a two-dimensional presentation gives a planar view, or “slice” through the body and shows the location and relative orientation of many features and characteristics within the body.

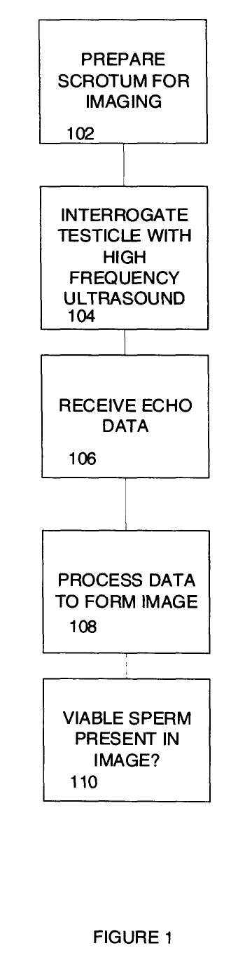

As shown in FIG. 1, in one exemplary aspect, a method for detecting viable sperm in a subject having at least one testicle comprises percutaneously interrogating (102, 104) at least a portion of the subject's testicle by transmitting sound having a frequency of at least about 15 megahertz (MHz) into the testicle. Echo data from the interaction of the transmitted sound and the testicle is received 106 and processed to form an image 108. At least one viable sperm can be detected in the image 110. As discussed above, a viable sperm detected in the image indicates viable sperm in the subject having at least one testicle. Optionally, any sperm can be detected by identifying at least one viable sperm in the image. Sperm can also be optionally detected in the image by identifying and sizing a seminiferous tubule in the image, wherein an increase in the size of the sized seminiferous tubule as compared to a control seminiferous tubule size indicates detection of viable sperm in the subject. The sizing of any portion of the tubule on the image can include use of a tracing and measurement functions that are common features of ultrasound imaging systems. These features can be automated, semi-automated or can be accomplished by a user of an ultrasound system. For example, these features can be used to determine the diameter of a tubule.

A control value can be from the same subject or can be from a different subject. Thus, a “control” can comprise either a tubule diameter measurement obtained from a control subject or can comprise a known standard. For example, a standard seminiferous tubule diameter can be determined for comparison to a measured diameter.

Thus, sperm can be detected in the image by identifying and sizing a seminiferous tubule in the image, wherein a tubule having a diameter of about 10 microns or greater indicates the detection of viable sperm. A tubule having a diameter of about 20 microns or greater can also indicate the detection of viable sperm. Moreover a tubule having a diameter of about, 30, 40, 50, 60, 70, 80, 90, 100, 110, 120, 130, 140, 150, 160, 170, 180, 190, 200, 210, 220, 230, 240, 250, 260, 270, 280, 290, or 300 microns or increments therebetween, or greater, can indicate the detection of viable sperm.

The received data can also include Doppler data. An increased perfusion indicated by the Doppler data can indicates the detection of viable sperm. Sperm can also be detected in the image by identifying movement of fluid within the testicle hilum.

Doppler ultrasound imaging methods and modes can be used to measure the velocity of blood flow or blood flow movement. The velocity or movement can be analyzed along with the measurements of tubule diameter to determine the presence of viable sperm. The Doppler measurements can be taken with the same high frequency ultrasound system used to produce the diameter measurements. Alternatively, a separate, high or clinical frequency ultrasound system could be used to produce blood flow velocity measurements.

Movement can be visualized by viewing a plurality of temporally distinct image frames. Such image frames can be viewed in sequential order as a cine loop. Sperm that has been identified using the systems and method can be removed from the subject.

Also provided is a method for screening a testicle for the presence of viable sperm, comprising percutaneously transmitting ultrasound having a frequency of at least 15 megahertz into the testicle. Echo data from the interaction of the transmitted ultrasound and the testicle is received. The received ultrasound is processed to provide an image. The presence of viable sperm in the screened testicle can be determined by detecting a viable sperm in the image to indicate the presence of viable sperm. In some aspects, no viable sperm are detected, which indicates an absence of viable sperm in the screened testicle. As described above, screening can comprise determining if viable sperm are present in the screened testicle by identifying and sizing a seminiferous tubule in the image. An increase in the size of the sized seminiferous as compared to a control seminiferous tubule size can indicate detection of viable sperm in the subject.

In another aspect, a method of predicting the success of vasectomy reversal in a subject comprises producing a percutaneous ultrasound image of at least a portion of the subject's vas deferens using ultrasound with a transmit frequency of at least 15 megahertz (MHz). Areas of vas deferens damage can be identified using the ultrasound image. The presence of damage in the vas deferens as compared to a control vas deferens indicating a diminished likelihood of success for vasectomy reversal in the subject. As described above, a control value can be from the same subject or can be from a different subject.

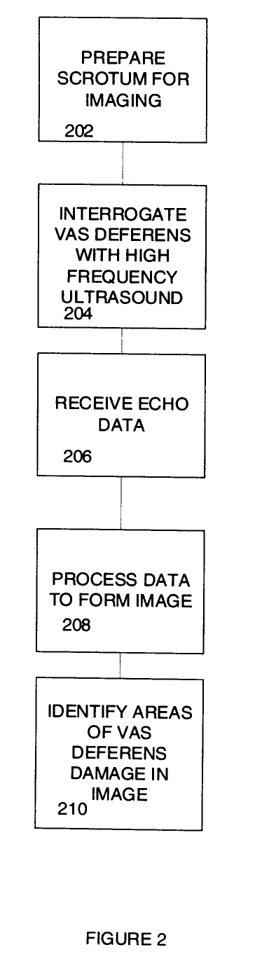

In one embodiment and as shown in FIG. 2, a method for identifying a damaged area of vas deferens in a subject comprises producing a percutaneous ultrasound image (202, 204, 206, 208) of at least a portion of the subject's vas deferens using ultrasound with a transmit frequency of at least 15 megahertz (MHz) and identifying an area of vas deferens damage using the ultrasound image 210. The area of damage can be identified by identifying an area of tubule obstruction. Optionally, the area of obstruction is identified by visualization of a dilated portion of a tubule, wherein the obstruction is located proximal to the dilated portion of the tubule. The area of obstruction can be identified by identifying and sizing a tubule in the image, wherein a tubule having a diameter of about 10 microns or greater indicates an area of obstruction. In some aspects, a tubule having a diameter of about 20 microns or greater indicates an area of obstruction. In other aspects, a tubule having a diameter of about 30, 40, 50, 60, 70, 80, 90, 100, 110, 120, 130, 140, 150, 160, 170, 180, 190, 200, 210, 220, 230, 240, 250, 260, 270, 280, 290, or 300 microns or increments therebetween or greater indicates an area of obstruction.

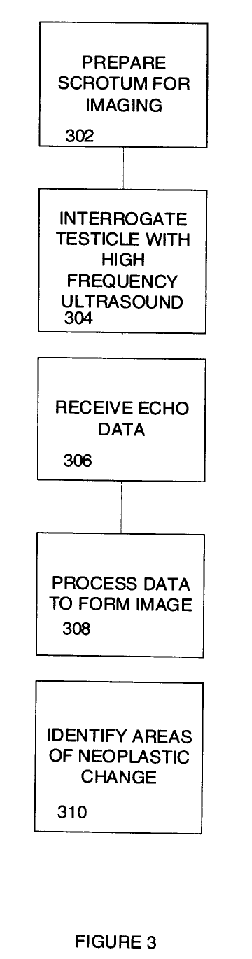

In a further embodiment and as shown in FIG. 3, a method for detecting neoplasia in a subject having at least one testicle comprises percutaneously interrogating 304 at least a portion of the subject's testicle by transmitting sound having a frequency of at least about 15 megahertz (MHz) into the testicle and receiving echo data 306 from the interaction of the transmitted sound and the testicle. The received data can be processed to form an image 308. A neoplastic change can be detected by analysis the image 310, wherein detected neoplastic changes indicated in the image indicates neoplasia in the subject having at least one testicle. For example, change indicated by alterations is vasculature in the testicle as compared to a control testicle can indicate a neoplastic change. An increase in vasculature in the testicle as compared to a control testicle or a change in the architecture of a seminiferous tubule as compared to a control seminiferous tubule can also indicate neoplastic change.

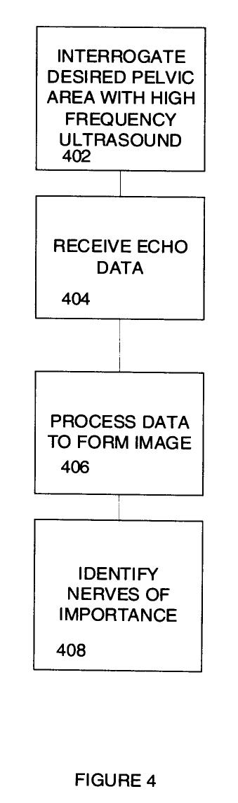

In a further embodiment, as shown in FIG. 4, a method for identifying a nerve in a subject comprises producing an ultrasound image (402, 404, 406) of at least a portion of the subject's nerve using ultrasound with a transmit frequency of at least about 15 megahertz (MHz) and identifying the nerve using the ultrasound image 408.

In an additional embodiment, a method for identifying small vessel anatomy in the penis of a subject comprises producing an ultrasound image (502, 504, 506) of at least a portion of the subject's penile small vasculature using ultrasound with a transmit frequency of at least about 15 megahertz (MHz) and identifying the small vasculature using the ultrasound image 508.

In conjunction with each of the described methods, an ultrasound contrast agent can be delivered to subject and imaging a described urologic structure comprising the administered ultrasound contrast agent can be performed.

A contrast agent for use in the disclosed methods can comprise a thin flexible or rigid shell composed of albumin, lipid or polymer confining a gas such as nitrogen or a perfluorocarbon. Other examples of representative gases include air, oxygen, carbon dioxide, hydrogen, nitrous oxide, inert gases, sulfur fluorides, hydrocarbons, and halogenated hydrocarbons, perfluorobutane, perfluoropropane, and sulfur hexafluoride. Liposomes or other microbubbles can also be designed to encapsulate gas or a substance capable of forming gas.

Administration of contrast imaging agents can be carried out in various fashions using a variety of dosage forms. For example, contrast agents can be injected directly into the urologic structures described. One preferred route of administration is intravascularly. For intravascular use, the contrast agent can be injected intravenously, but can be injected intra-arterially as well. The useful dosage to be administered and the mode of administration can vary depending upon the age and weight of the subject, and on the particular diagnostic application intended. In one aspect, a dosage can be initiated at lower levels and increased until the desired contrast enhancement is achieved. A contrast agent can be administered in the form of an aqueous suspension such as in water or a saline solution (e.g., phosphate buffered saline). In this aspect, the water can be sterile and the saline solution can be a hypertonic saline solution (e.g., about 0.3 to about 0.5% NaCl), although, if desired, the saline solution can be isotonic. Optionally, the solution also can be buffered, if desired, to provide a pH range of pH 6.8 to pH 7.4. In addition, dextrose can be included in the media.

The desired ultrasound for use with the disclosed methods can be applied, transmitted and received using an ultrasonic scanning device that can supply ultrasound at a center frequency sufficient to accurately resolve the desired structures to be imaged. For example, a system with a center frequency transmit of at least about 15 MHz to the highest practical frequency can be used. In exemplary aspects, ultrasound can be supplied at a center frequency of about 15 MHz, 20 MHz, 25 MHz, 30 MHz, 35 MHz, 40 MHz, 45 MHz, 50 MHz, 55 MHz, 60 MHz, 65 MHz, 70 MHz, or higher, and at frequencies therebetween. Thus, an ultrasound system or device capable of operating at about 15 MHz or above can be used. The systems can achieve a bandwith of between about 10 MHz and about 80 MHz.

One such exemplary system is the VisualSonics™ (Toronto, Calif.) UBM system model VS40 VEVO™ 660. Another exemplary system is the VisualSonics™ (Toronto, Calif.) model VEVO™ 770.

Another such exemplary system can have the components and functionality described in U.S. patent application Ser. No. 10/683,890, US patent application publication 20040122319 and now U.S. Pat. No. 7,255,678, which is incorporated herein by reference.

It is contemplated that any system capable of producing an ultrasound image using a high frequency ultrasound can be used. Thus, the methods can be practiced using a mechanically scanned ultrasound system that can translate an ultrasound beam as it sweeps along a path. The methods can also be practiced using an array based system where the beam is translated by electrical steering of an ultrasound beam along the elements of the transducer. One skilled in the art will readily appreciate that beams translated from either type system can be used in the described methods, without any limitation to the type of system employed. The type of system is therefore not intended to be a limitation to any described method because array and mechanically scanned systems can be used interchangeably to perform the described methods.

In various embodiments, a system of the present invention for detecting viable sperm in a subject having at least one testicle, for screening a testicle for the presence of viable sperm, for predicting the success of vasectomy reversal in a subject, for identifying a damaged area of vas deferens in a subject, for detecting neoplasia in a subject having at least one testicle, for identifying a nerve in a subject, and for identifying small vessel anatomy in the penis of a subject can comprise means for interrogating at least a portion of the subject's testicle or urologic structure of interest by transmitting sound having a frequency of at least about 15 megahertz (MHz) into the testicle or structure of interest and means for receiving echo data from the interaction of the transmitted sound and the testicle or structure of interest.

Such means can include an ultrasonic imaging probe including a mechanically scanned transducer or an array transducer. The probe/transducer can be operatively connected to a processing system or means for processing the received data to form an image. The processing means or another processing means can display the image and at least one viable sperm or structural characteristic in the image can be detected. For example, a viable sperm detected in the image can indicate viable sperm in the subject having at least one testicle. The processing can include automated comparison of measured data to a control as described above.

An exemplary ultrasound system can comprise software for producing an ultrasound image, for analyzing blood flow data, for taking and analyzing size measurements, and for identifying structures or features comprising the image. Such software can comprise an ordered listing of executable instructions for implementing logical functions, and can be embodied in any computer-readable medium for use by or in connection with an instruction execution system, apparatus, or device, such as a computer-based system, processor-containing system, or other system that can fetch the instructions from the instruction execution system, apparatus, or device and execute the instructions.

For example, a control value can be stored in the ultrasound system in a computer readable code or medium or can be similarly stored in a separate computational device. Software of the ultrasound system or computational device can compare a measured tubule diameter to a control value and can determine whether tubule dilatation is present. Thus, the system can comprise computer readable code and a processor for determining a tubule dilatation from a captured image.

In the context of this document, a “computer-readable medium” can be any means that can contain, store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium.

More specific examples (a non-exhaustive list) of the computer-readable medium would include the following: an electrical connection (electronic) having one or more wires, a portable computer diskette (magnetic), a random access memory (RAM), a read-only memory (ROM), an erasable programmable read-only memory (EPROM or Flash memory) (magnetic), an optical fiber (optical), and a portable compact disc read-only memory (CDROM) (optical). Note that the computer-readable medium could even be paper or another suitable medium upon which the program is printed, as the program can be electronically captured, via for instance optical scanning of the paper or other medium, then compiled, interpreted or otherwise processed in a suitable manner if necessary, and then stored in a computer memory.

An exemplary imaging system can include memory. Memory can include the image data obtained by an ultrasound system. A computer readable storage medium can be coupled to the processor for providing instructions to the processor to instruct and/or configure processor to perform steps or algorithms related to the operation of the ultrasound system, including algorithms related to the measurement of urologic structures or to analysis of blood flow velocity.

The computer readable medium can include hardware and/or software such as, by way of example only, magnetic disks, magnetic tape, optically readable medium such as a CD ROM, and semiconductor memory such as a PCMCIA card. In each case, the medium may take the form of a portable item such as a small disk, floppy diskette, cassette, or it may take the form of a relatively large or immobile item such as hard disk drive, solid state memory card, or RAM provided in the support system. It should be noted that the above listed example mediums can be used either alone or in combination.

### EXAMPLES

The following examples are put forth so as to provide those of ordinary skill in the art with a complete disclosure and description of how the compositions, articles, devices, systems, and/or methods claimed herein are made and evaluated, and are intended to be purely exemplary and are not intended to limit the scope of compositions, compositions, articles, devices, systems, and/or methods. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

Hair is removed from the scrotum and a warm gel is placed on the scrotum overlying a testicle. Ultrasound scanning begins by placing the probe on the gelled area of the scrotum overlying the testicle. The testicle and epididymis is interrogated using high frequency ultrasound.

Seminiferous tubules are located and the largest or most obvious tubule is identified and capture in a cine loop. The largest seminiferous tubule is imaged and measured and store as an image or loop.

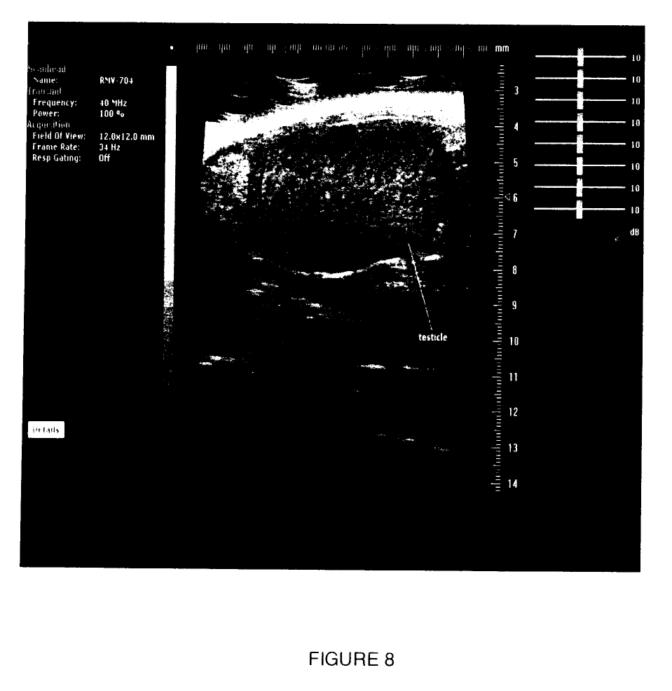

FIG. 8 is an image of a mouse testicle in longitudinal axis. The image was produced using a VisualSonics Inc. RMV™ 704 (40 MHz) probe.

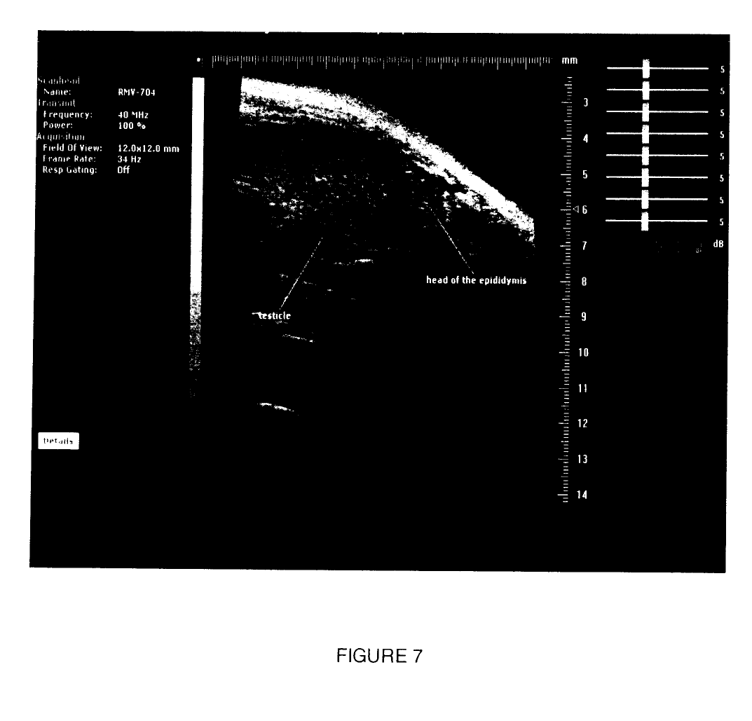

FIG. 7 shows identification of the head of the epididymis in an exemplary ultrasound image. A VisualSonics Inc. RMV™ 704 (40 MHz) probe was used to create the image. The testicle was located in the longitudinal axis and the probe was moved proximally along testicle to locate the head of the epididymis. Using the image, sperm can be identified and areas of obstruction and damage can be identified. Identification of sperm in the epididymis can indicate presence of viable sperm, which can be quantified.

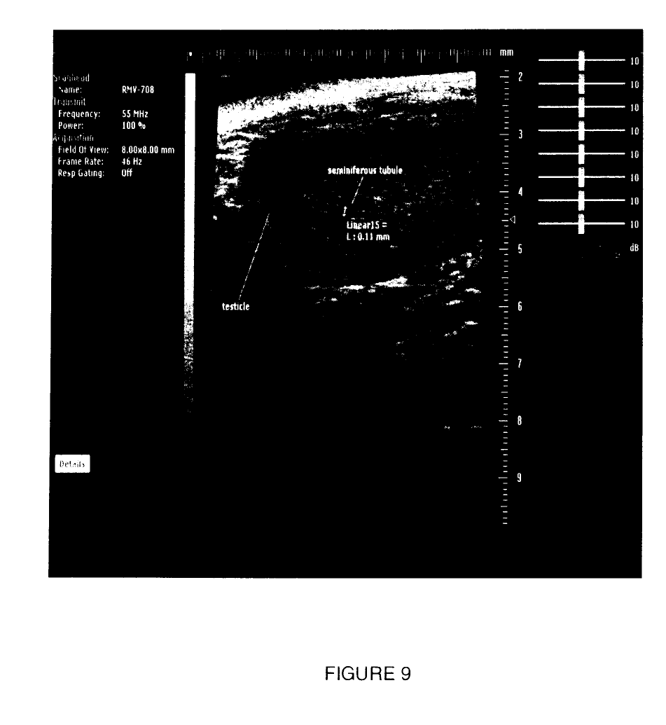

FIG. 9 shows a high frequency ultrasound image indicating a seminiferous tubule in the testicle. A VisualSonics Inc. RMV™ 708 (55 MHz) probe was used to produce the image. The testicle was located in a longitudinal axis and seminiferous tubules were identified in an image by locating linear ducts that run through the testicle, which are the seminiferous tubules. Sperm can be identified in the tubules and they can be exemplarily extracted with a needle.

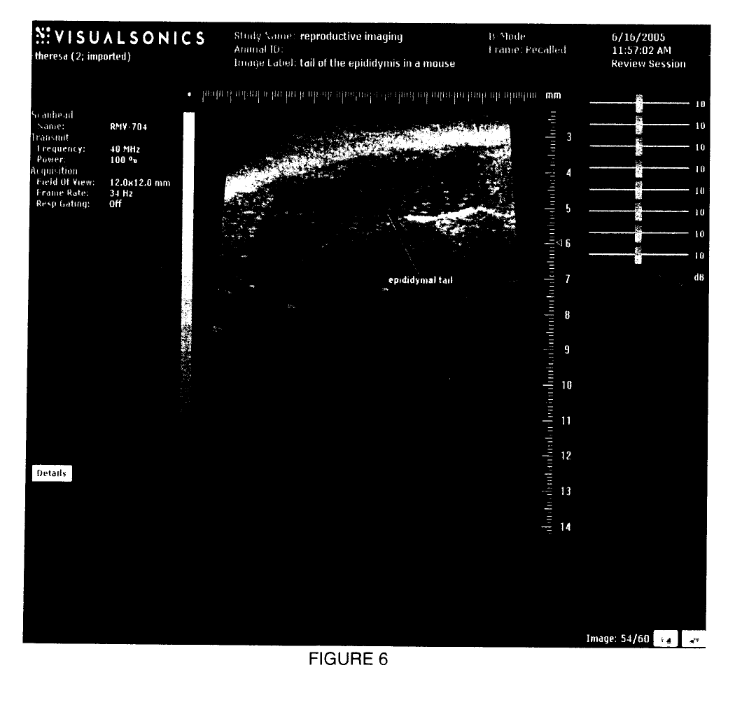

FIG. 6 is an image of the tail of the epididymis in a mouse created using a VisualSonics Inc. RMV™ 704 (40 MHz) probe. The testicle was located in a longitudinal axis and then the probe was moved lateral to the testicle. The epididymal structure was identified by determining a structure starting proximal to the testicle and lying laterally to the testicle and travels the length of the testicle. Sperm can be identified in the epididymal structure and damage can also be identified.

### Example 2

Hair is removed from the scrotum and a warm gel is placed on the scrotum overlying a testicle. Ultrasound scanning begins by placing the probe on the gelled area of the scrotum overlying the testicle. The testicle and epididymis is interrogated using high frequency ultrasound. The vas deferens is identified and the suture/clip where the vasectomy was performed is located.

### Example 3

The high-resolution micro ultrasound (Vevo) is used establish normal (control) parameters for human testis. The Vevo system can detect spatial difference down to 30 μm or 10⁻⁶ m. This system can be used to evaluate testicular tubules (200 μm) and sperm (50 μm), which is not possible using the standard ultrasound.

Twenty fertile (previous proven paternity) male subjects aged 18 and older with no significant medical or surgical history and no history of testicular trauma or congenital abnormalities are evaluated. High resolution ultrasound is used to determine seminiferous tubule size including the diameter of tubules and lumen (mean and SD). Seminiferous tubular fluid flow and movement of spermatozoa are also evaluated. Additional features evaluated include vascular distribution—arterial flow, resistive index, venous drainage distribution, retes testis—size, fluid flow, sperm movement, efferent ductules—diameter, fluid flow, sperm movement and Epididymus—diameter of tubules and lumen, fluid flow, sperm movement in the head, body and tail. The vas deferens is also evaluated including the diameter of tubules and lumen, fluid flow and sperm in the convoluted portion and straight portion.

The determined dimensions of the normal human testis are approximately 4-5 cm in length, and 2.5-3 cm in width. The diameter of individual seminiferous tubules is determined using high frequency ultrasound to be about 180-280 um. Each seminiferous tubule with normal spermatogenesis is lined by a layer of seminiferous epithelium including Sertoli cells, and germ cells at various developing stages and is determined to be about 60-80 um. The measured luminal diameter is about 20 to 160 um.

High frequency ultrasound is used to differentiate between patients with Obstructive (OA) versus Non-obstructive azoospermia (NOA). NOA patient with Sertoli Cell Only (SCO) pattern or early maturation arrest (MA) have atrophic tubules, or fibrotic tubules with no apparent lumen. Clinically, these patients have small testis and elevated follicular stimulating hormone (FSH) level in the serum. However, it is difficult to differentiate patient with late maturation arrest (MA) or hypospermatogenesis from OA on physical examination and serum gonadotropins.

Currently, an invasive testicular biopsy is required to make the diagnosis of OA or NOA. Although the tubular diameter can be equivalent between late MA or hypospermatogenic to the OA patient, the luminal diameter can also be enlarged in the OA seminiferous tubules due to back pressure from the obstruction. In addition, there can be a point of transition in the testis or epididymus of the OA testis, which is not be apparent in the NOA testis.

Fifty male patients aged 18 and older with azoospermia (no sperm in the ejaculation) are evaluated. Using high frequency ultrasound both NOA (late MA and hypospermatogenesis) and OA testis seminiferous tubular diameter is determined to be similar to the normal controls (180-280 um). The luminal diameter for the OA group is larger than control group (100-200 um) and can be used to distinguish OA from NOA.

### Example 4

High frequency ultrasound is used to establish a prognostic parameter for patients with Non-obstructive azoospermia (NOA) undergoing microdissection testicular sperm extraction (micro-TESE). Micro-TESE is an operative procedure using a microscope to locate seminiferous tubules that may contain mature sperm in the testis. This operation is under general anesthesia and may take up to 2-3 hours. The success rate for viable sperm retrieval using this technique is approximately 50%, and currently there is no prognostic tool to identify which patients will fail.

A non-invasive ultrasound based tool is used to measure the seminiferous tubular size, and optionally, to visualize sperm movement. The predetermined location of normal tubules on ultrasound can also guide surgeon to find the “optimal” site for locating viable sperm and to decrease operating time.

Subjects age 18 and older having non-obstructive azoospermic are evaluated. Subjects having NOA are diagnosed on either previous testicular biopsy, or clinical finding of small testis with elevated FSH. Patients who are scheduled to undergo micro-TESE for the purpose of intracytoplasmic sperm injection (ICSI) are evaluated using high frequency ultrasound imaging. In testis with no viable spermatozoa, the seminiferous tubules appear to be atrophic (20-50 um in diameter) throughout the entire testis. In the testis containing mature spermatozoa, there are patches or island of enlarged tubules with diameter ranging from 150 to 280 um, which is similar or slightly smaller than the normal control.

The preceding description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. The corresponding structures, materials, acts, and equivalents of all means or step plus function elements in the claims below are intended to include any structure, material, or acts for performing the functions in combination with other claimed elements as specifically claimed.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification. The blocks in the flow charts described above can be executed in the order shown, out of the order shown, or substantially in parallel.

Various publications were referenced in preparation of this application. The disclosures of these publications in their entireties are hereby incorporated by reference into this application in order to more fully describe the state of the art to which this invention pertains.

Accordingly, those who work in the art will recognize that many modifications and adaptations to the present invention are possible and can even be desirable in certain circumstances and are a part of the present invention. Other embodiments of the invention will be apparent to those skilled in the art from consideration of the specification and practice of the invention disclosed herein. Thus, the preceding description is provided as illustrative of the principles of the present invention and not in limitation thereof. It is intended that the specification and examples be considered as exemplary only, with a true scope and spirit of the invention being indicated by the following claims.

1. A method for detecting viable sperm in a subject having at least one testicle, comprising:
percutaneously interrogating at least a portion of the subject's testicle by transmitting sound having a frequency of at least about 15 megahertz (MHz) into the testicle;
receiving echo data from the interaction of the transmitted sound and the testicle;
processing the received data to form an image; and
determining if viable sperm are present by detecting at least one viable sperm in the image, wherein a viable sperm detected in the image indicates viable sperm in the subject having at least one testicle.

2. The method of claim 1, wherein the sperm is detected by identifying at least one viable sperm in the image.

3. The method of claim 1, wherein the sperm is detected in the image by identifying and sizing a seminiferous tubule in the image, wherein an increase in the size of the sized seminiferous as compared to a control seminiferous tubule size or a tubule having a diameter of about 10 microns or greater indicates detection of viable sperm in the subject.

4. (canceled)

5. The method of claim 3, wherein a tubule having a diameter of about 20 microns or greater indicates the detection of viable sperm.

6. (canceled)

7. The method of claim 1, wherein the received data includes Doppler data and wherein increased perfusion indicated by the Doppler data indicates the detection of viable sperm.

8. The method of claim 1, wherein the sperm is detected in the image by identifying movement of fluid within the testicle hilum.

9. The method of claim 1, further comprising percutaneously removing identified viable sperm from the subject.

10. (canceled)

11. The method of claim 1, further comprising percutaneously harvesting identified sperm.

12. The method of claim 1, wherein no viable sperm are detected to indicate an absence of viable sperm in the screened testicle

13. (canceled)

14. (canceled)

15. A method for screening a testicle for the presence or absence of viable sperm, comprising:
percutaneously transmitting ultrasound having a frequency of at least 15 megahertz into the testicle; receiving echo data from the interaction of the transmitted ultrasound and the testicle; processing the received ultrasound to provide an image; identifying a seminiferous tubule or portion thereof in the ultrasound image; and determining the lumen diameter of the identified seminiferous tubule wherein an increase in the lumen diameter of the identified seminiferous tubule as compared to a control seminiferous tubule lumen diameter indicates detection of viable sperm in the testicle.

16. (canceled)

17. The method of claim 15, wherein a lumen diameter of the identified seminiferous tubule of between about 20 microns and about 400 microns indicates a seminiferous tubule comprising viable sperm in the testicle.

18. (canceled)

19. (canceled)

20. (canceled)

21. The method of claim 15, wherein an increase in the lumen diameter of the identified seminiferous tubule as compared to a control seminiferous tubule lumen diameter indicates detection of viable sperm in the testicle.

22. The method of claim 15, wherein a decrease or no change in the lumen diameter of the identified seminiferous tubule as compared to a control seminiferous tubule lumen diameter indicates the non-detection of viable sperm in the subject.

23. The method of claim 15, wherein
an increase in the lumen diameter of the identified seminiferous tubule as compared to a control seminiferous tubule lumen diameter indicates obstructive azoospermia.

24. (canceled)

25. The method of claim 23, wherein a lumen diameter of the identified seminiferous tubule of between about 20 microns and about 400 microns indicates obstructive azoospermia.

26. (canceled)

27. (canceled)

28. (canceled)

29. A system for detecting viable sperm in a subject having at least one testicle, comprising:
means for interrogating at least a portion of the subject's testicle by transmitting sound having a frequency of at least about 15 megahertz (MHz) into the testicle;
means for receiving echo data from the interaction of the transmitted sound and the testicle; means for processing the received data to form an image; and
means for detecting at least one viable sperm in the image, wherein a viable sperm detected in the image indicates viable sperm in the subject having at least one testicle.

30. The system of claim 29, further comprising means for identifying at least one viable sperm in the image.

31. The system of claim 29, further comprising means for identifying and sizing a seminiferous tubule in the image, wherein an increase in the size of the sized seminiferous as compared to a control seminiferous tubule size indicates detection of viable sperm in the subject.

32. (canceled)

33. A method for identifying a damaged area of vas deferens in a subject, comprising:
producing a percutaneous ultrasound image of at least a portion of the subject's vas deferens using ultrasound with a transmit frequency of at least 15 megahertz (MHz); and
identifying an area of vas deferens damage using the ultrasound image.

34. The method of claim 33, wherein the area of damage is identified by identifying an area of tubule obstruction.

35. The method of claim 34, wherein the area of obstruction is identified by visualization of a dilated portion of a tubule, wherein the obstruction is located proximal to the dilated portion of the tubule.

36. The method of claim 35, wherein the area of obstruction is identified by identifying and sizing a tubule in the image, wherein a tubule having a diameter of about 10 microns or greater indicates an area of obstruction.

37. The method of claim 35, wherein a tubule having a diameter of about 20 microns or greater indicates an area of obstruction.

38. (canceled)

39. (canceled)

40. A system for identifying a damaged area of vas deferens in a subject, comprising: means for producing a percutaneous ultrasound image of at least a portion of the subject's vas deferens using ultrasound with a transmit frequency of at least 15 megahertz (MHz); and means for identifying an area of vas deferens damage using the ultrasound image.

41. A method for detecting neoplasia in a subject having at least one testicle, comprising:
percutaneously interrogating at least a portion of the subject's testicle by transmitting sound having a frequency of at least about 15 megahertz (MHz) into the testicle; receiving echo data from the interaction of the transmitted sound and the testicle;
processing the received data to form an image; and
detecting a neoplastic change indicated in the image, wherein detected neoplastic changes indicated in the image indicates neoplasia in the subject having at least one testicle.

42. The method of claim 41, wherein the detected neoplastic change is indicated by alterations in vasculature in the testicle as compared to a control testicle.

43. (canceled)

44. The method of claim 41, wherein the detected neoplastic change is indicated by a change in the architecture of a seminiferous tubule as compared to a control seminiferous tubule.

45. A method for identifying a nerve in a subject, comprising: producing an ultrasound image of at least a portion of the subject's nerve using ultrasound with a transmit frequency of at least about 15 megahertz (MHz); and determining the presence of a nerve in the ultrasound image to identify a nerve in the subject.

46. A method for identifying small vessel anatomy in the penis of a subject, comprising: producing an ultrasound image of at least a portion of the subject's penile small vasculature using ultrasound with a transmit frequency of at least about 15 megahertz (MHz); and determining the presence of a small vessel in the image to identify small vessel anatomy in the penis of the subject.
